(19) United States
(12) Patent Application Publication
(10) Pub. No.: US 2009/0280566 A1
(43) Pub. Date: Nov. 12, 2009

(54) METHODS FOR INCREASING GERMINATION FREQUENCY AND/OR VIGOR BY COLD SHOCK TREATMENT OF CONIFER SOMATIC EMBRYOS DURING DEVELOPMENT

(75) Inventors: CAROLYN V. CARPENTER, SEATTLE, WA (US); MAYUMI IKEDA, TACOMA, WA (US)

Correspondence Address: WEYERHAEUSER COMPANY;INTELLECTUAL PROPERTY DEPT., CH 1J27, P.O. BOX 9777, FEDERAL WAY, WA, 98063, US

(73) Assignee: WEYERHAEUSER NR COMPANY, FEDERAL WAY, WA (US)

(21) Appl. No.: 12/433,048

(22) Filed: Apr. 30, 2009

Related U.S. Application Data
(60) Provisional application No. 61/051,553, filed on May 8, 2008.

(51) Int. Cl. C12N 5/04

(52) U.S. Cl. 435/422

(57) ABSTRACT

In one aspect, a method is provided for increasing germination vigor and/or frequency of conifer somatic embryos produced in vitro. The method comprises (a) incubating a plurality of immature conifer somatic embryos for a first incubation period in, or on, a first development media at a temperature in the range of 20° C. to 30° C.; (b) exposing the plurality of immature conifer somatic embryos incubated in accordance with step (a) to a cold temperature in the range of 0° C. to 10° C. for a time period of at least one week; and (c) incubating the plurality of immature conifer somatic embryos treated in accordance with step (b) for a second incubation period in, or on, a second development medium at a temperature in the range of 20° C. to 30° C.

### FIELD OF THE INVENTION

The present invention relates to methods for increasing germination frequency and/or vigor by cold shock treatment of conifer somatic embryos during development.

### BACKGROUND

The demand for coniferous trees, such as pines and firs, to make wood products continues to increase. One proposed solution to the problem of providing an adequate supply of coniferous trees is to identify individual coniferous trees that possess desirable characteristics, such as a rapid rate of growth, and to produce numerous, genetically identical, clones of the superior trees by somatic cloning.

Somatic cloning is the process of creating genetically identical trees from tree somatic tissue. Tree somatic tissue is tree tissue other than the male and female gametes. In one approach to somatic cloning, tree somatic tissue is cultured in an initiation medium which includes hormones, such as auxins and/or cytokinins that initiate formation of embryogenic cells that are capable of developing into somatic embryos. The embryogenic cells are then further cultured in a maintenance medium that promotes multiplication of the embryogenic cells to form pre-cotyledonary embryos (i.e., embryos that do not possess cotyledons). The multiplied embryogenic cells are then cultured in a development medium that promotes development and maturation of cotyledonary somatic embryos which can, for example, be placed within artificial seeds and sown in the soil where they germinate to yield conifer seedlings. The seedlings can be transplanted to a growth site for subsequent growth and eventual harvesting to yield lumber, or wood-derived products. Alternatively, the cotyledonary somatic embryos can also be germinated in a germination medium, and thereafter transferred to soil for further growth.

A continuing problem with somatic cloning of conifer embryos is stimulating efficient and cost-effective formation of somatic embryos that are capable of germinating to yield plants. Preferably, conifer somatic embryos, formed in vitro, are physically and physiologically similar, or identical, to conifer zygotic embryos formed in vivo in conifer seeds. There is, therefore, a continuing need for methods for producing viable conifer somatic embryos from conifer embryogenic cells.

### SUMMARY

In one aspect, a method is provided for increasing germination frequency and/or vigor of conifer somatic embryos produced in vitro. The method comprises (a) incubating a plurality of immature conifer somatic embryos for a first incubation period in, or on, a first development media at a temperature in the range of 20° C. to 30° C.; (b) exposing the plurality of immature conifer somatic embryos incubated in accordance with step (a) to a cold temperature in the range of 0° C. to 10° C. for a time period of at least one week; and (c) incubating the plurality of immature conifer somatic embryos treated in accordance with step (b) for a second incubation period in, or on, a second development medium at a temperature in the range of 20° C. to 30° C.

In another aspect, a method is provided for producing mature conifer somatic embryos. The method comprises (a) culturing conifer somatic cells in, or on, an induction medium to yield embryogenic cells; (b) culturing the embryogenic cells prepared in step (a) in, or on, a maintenance medium to multiply the embryogenic cells and form pre-cotyledonary conifer somatic embryos; (c) culturing the pre-cotyledonary conifer somatic embryos formed in step (b) in, or on, a first development medium at a temperature in the range of 20° C. to 30° C. for a first incubation period; (d) exposing the plurality of immature conifer somatic embryos incubated in accordance with step (c) to a cold temperature in the range of 0° C. to 10° C. for a time period of at least one week; and (e) incubating the plurality of immature conifer somatic embryos treated in accordance with step (d) for a second incubation period in, or on, a second development medium at a temperature in the range of 20° C. to 30° C.

The methods of the present invention are useful for preparing mature conifer somatic embryos with increased germination frequency and/or vigor that can be further characterized, such as by genetic or biochemical means, and/or can be germinated to produce conifers, if so desired. Thus, for example, the methods of the invention can be used to more efficiently produce clones of individual conifers that possess one or more desirable characteristics, such as rapid growth rate or improved wood quality.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatic representation of early and late stage development of conifer somatic embryos;

FIG. 2 is a flow diagram illustrating an embodiment of a method of increasing germination vigor and/or frequency of somatic embryos;

FIG. 3A graphically illustrates the germination fraction (category 1+2) for genotype A somatic pine embryos that were or were not exposed to a cold treatment at various time points during development, as described in TABLE 3;

FIG. 3B graphically illustrates the germination fraction (category 1+2) for genotype B somatic pine embryos that were or were not exposed to a cold treatment at various time points during development, as described in TABLE 3;

FIG. 3C graphically illustrates the germination fraction (category 1+2) for genotype C somatic pine embryos that were or were not exposed to a cold treatment at various time points during development, as described in TABLE 3;

FIG. 3D graphically illustrates the germination fraction (category 1+2) for genotype D somatic pine embryos that were or were not exposed to a cold treatment at various time points during development, as described in TABLE 3;

FIG. 4A graphically illustrates the germination fraction (category 1+2) of two genotypes of somatic pine embryos that were exposed to a cold treatment at either 6 weeks or 8 weeks into development, or no cold treatment during development, followed by stratification or no stratification;

FIG. 4B graphically illustrates the germination fraction (category 1+2) of two genotypes of somatic pine embryos that were exposed to a cold treatment at either 6 weeks or 8 weeks into development, or no cold treatment during development, with singulation at either 8 weeks or after development;

FIG. 5A graphically illustrates the germination fraction (category 1) for genotype A somatic pine embryos that were or were not exposed to a cold treatment at various time points during development, as described in TABLE 3;

FIG. 5B graphically illustrates the germination fraction (category 1) for genotype B somatic pine embryos that were or were not exposed to a cold treatment at various time points during development, as described in TABLE 3;

FIG. 5C graphically illustrates the germination fraction (category 1) for genotype C somatic pine embryos that were or were not exposed to a cold treatment at various time points during development, as described in TABLE 3;

FIG. 5D graphically illustrates the germination fraction (category 1) for genotype D somatic pine embryos that were or were not exposed to a cold treatment at various time points during development, as described in TABLE 3;

FIG. 6A graphically illustrates the germination fraction (category 1) for two genotypes of somatic pine embryos that were exposed to a cold treatment at either 6 weeks or 8 weeks into development, or no cold treatment during development, followed by stratification or no stratification;

FIG. 6B graphically illustrates the germination fraction (category 1) of two genotypes of somatic pine embryos that were exposed to a cold treatment at either 6 weeks or 8 weeks into development, or no cold treatment during development, with singulation at either 8 weeks or after development;

FIG. 7A graphically illustrates the distribution of root length (mm) for germinants of genotype A resulting from somatic embryos treated with or without a cold treatment during development, as described in TABLE 3;

FIG. 7B graphically illustrates the distribution of root length (mm) for germinants of genotype B resulting from somatic embryos treated with or without a cold treatment during development, as described in TABLE 3;

FIG. 7C graphically illustrates the distribution of root length (mm) for germinants of genotype C resulting from somatic embryos treated with or without a cold treatment during development, as described in TABLE 3;

FIG. 7D graphically illustrates the distribution of root length (mm) for germinants of genotype D resulting from somatic embryos treated with or without a cold treatment during development, as described in TABLE 3;

FIG. 8A graphically illustrates the root length of germinants resulting from two genotypes of somatic pine embryos that were exposed to a cold treatment at either 6 weeks or 8 weeks into development, or no cold treatment during development, with singulation at either 8 weeks or after development, with no stratification;

FIG. 8B graphically illustrates the root length of germinants resulting from two genotypes of somatic pine embryos that were exposed to a cold treatment at either 6 weeks or 8 weeks into development, or no cold treatment during development, with singulation at either 8 weeks or after development, followed by stratification;

FIG. 9A graphically illustrates the hypocotyl length of germinants (category 1+2) of genotype A resulting from somatic embryos treated with or without a cold treatment during development, as described in TABLE 3;

FIG. 9B graphically illustrates the hypocotyl length of germinants (category 1+2) of genotype B resulting from somatic embryos treated with or without a cold treatment during development, as described in TABLE 3;

FIG. 9C graphically illustrates the hypocotyl length of germinants (category 1+2) of genotype C resulting from somatic embryos treated with or without a cold treatment during development, as described in TABLE 3;

FIG. 9D graphically illustrates the hypocotyl length of germinants (category 1+2) of genotype D resulting from somatic embryos treated with or without a cold treatment during development, as described in TABLE 3;

FIG. 10A graphically illustrates the hypocotyl length of germinants resulting from two genotypes of somatic pine embryos that were exposed to a cold treatment at either 6 weeks or 8 weeks into development, or no cold treatment during development, with stratification or without stratification; and

FIG. 10B graphically illustrates the hypocotyl length of germinants resulting from two genotypes of somatic pine embryos that were exposed to a cold treatment at either 6 weeks or 8 weeks into development, or no cold treatment during development, with singulation at either 8 weeks or after development.

### DETAILED DESCRIPTION

Unless specifically defined herein, all terms used herein have the same meaning as they would to one skilled in the art of the present invention.

As used herein, the term “development stage” refers to the period during somatic cloning during which histogenesis and growth of tissues and organs occurs in an immature embryo to reach a full-sized mature embryo capable of germination into a plant.

As used herein, the term “immature embryo” refers to an embryo that is not yet capable of germination into a plant, and includes embryos in early stage development (i.e., pre-cotyledonary embryos), and mid-stage development (i.e., embryos with cotyledons or hypocotyls that are not yet fully developed).

As used herein, the term “anatomical maturity” refers to an embryo that possesses developed cotyledons and hypocotyl.

As used herein, the term “cotyledonary embryo” refers to an embryo with a well-defined, elongated bipolar structure with latent meristematic centers having one or more clearly visible cotyledonary primordia at one end and a latent radicle at the opposite end.

As used herein, the term “pre-cotyledonary embryo” refers to an embryo that does not yet have cotyledons.

As used herein, the term “normal germinant” denotes the presence of all expected parts of a plant at time of evaluation. The expected parts of a plant may include a radicle, a hypocotyl, one or more cotyledon(s), and an epicotyl. In the case of gymnosperms, a normal germinant is characterized by the radicle having a length greater than 3 mm and no visibly discernable malformations compared to the appearance of embryos germinated from natural seed.

As used herein, the term “radicle” refers to the part of a plant embryo that develops into the primary root of the resulting plant.

As used herein, the term “hypocotyl” refers to the portion of a plant embryo or seedling located below the cotyledons but above the radicle.

As used herein, the term “epicotyl” refers to the portion of the seedling stem that is above the cotyledons.

As used herein, the term “embryonal suspensor mass” or “ESM” refers to a cell mass plated onto the surface of nutrient medium contained either in a semi-solid gel or as a liquid in a porous matrix capable of providing physical support, and left to grow for a period up to three months. During the three month incubation time, somatic embryos grow from microscopic precursor cell groups into visible early-stage embryos and eventually to anatomically mature embryos. The structure of the ESM after several weeks of incubation typically consists of a proliferated mat with a few embryos sitting in direct contact with media, but most embryos forming on the top or side of the still proliferating cell mass.

As used herein, the term “germination frequency” refers to the number, proportion, percentage or fraction of germinants in a particular population of somatic embryos.

Unless stated otherwise, all concentration values that are expressed as percentages are weight per volume percentages.

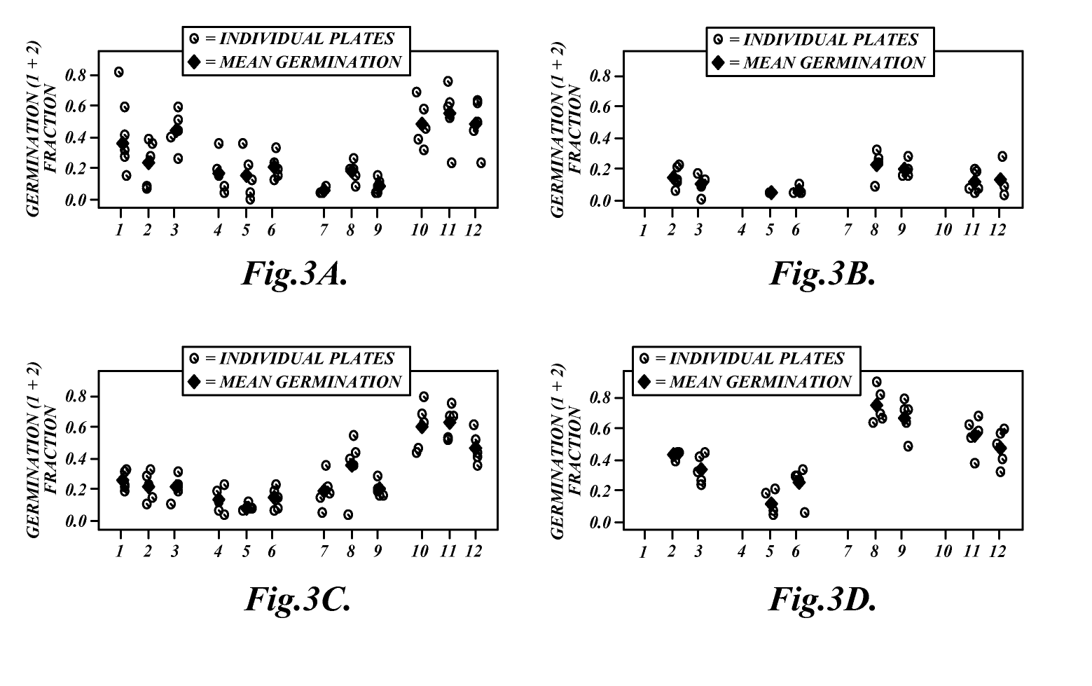

In accordance with the methods of the invention, it has been unexpectedly discovered by the present inventors that exposing immature conifer somatic embryos during the development stage to a cold temperature in the range of 0° C. to 10° C. for a time period of at least one week, followed by incubating the somatic embryos on development medium at a temperature in the range of 20° C. to 30° C. for an additional period of time, produces embryos that germinate at an increased frequency and/or have improved vigor, as compared to embryos that are not exposed to a cold temperature during development, as described in Example 2 and shown in FIGS. 3A-10B.

In accordance with the foregoing, in one aspect, a method is provided for increasing germination frequency and/or vigor of conifer somatic embryos produced in vitro. The method comprises (a) incubating a plurality of immature conifer somatic embryos for a first incubation period in, or on, a first development media at a temperature in the range of 20° C. to 30° C.; (b) exposing the plurality of immature conifer somatic embryos incubated in accordance with step (a) to a cold temperature in the range of 0° C. to 10° C. for a time period of at least one week; and (c) incubating the plurality of immature conifer somatic embryos treated in accordance with step (b) for a second incubation period in, or on, a second development medium at a temperature in the range of 20° C. to 30° C.

The methods of the invention can be used to produce cotyledonary somatic embryos from any conifer, such as members of the genus Pinus, such as Loblolly pine (Pinus taeda) and Radiata pine. For example, as described in a journal article by R. Nagamani et al., entitled “Anatomical Comparison of Somatic and Zygotic Embryogeny in Conifers,” in S. M. Jain et al. (eds), Vol. 1, Somatic Embryogenesis in Woody Plants, Series: Forestry Sciences, Vol. 44, 1995, pp. 23-48, a reasonable correlation is expected to exist between the culturing of embryos from Loblolly Pine and other pine species because zygotic and somatic embryos of the genus Pinus are anatomically similar and recognized as having similar embryogenic potentials in culture media. Again, by way of example, Douglas fir embryos can be produced by the methods of the invention.

A population of mature conifer somatic embryos produced according to the methods of the invention has a greater frequency of germinating into conifer plants than a population of conifer somatic embryos produced according to an otherwise identical control method that does not include the step of exposing the immature embryos to a cold treatment during the development stage.

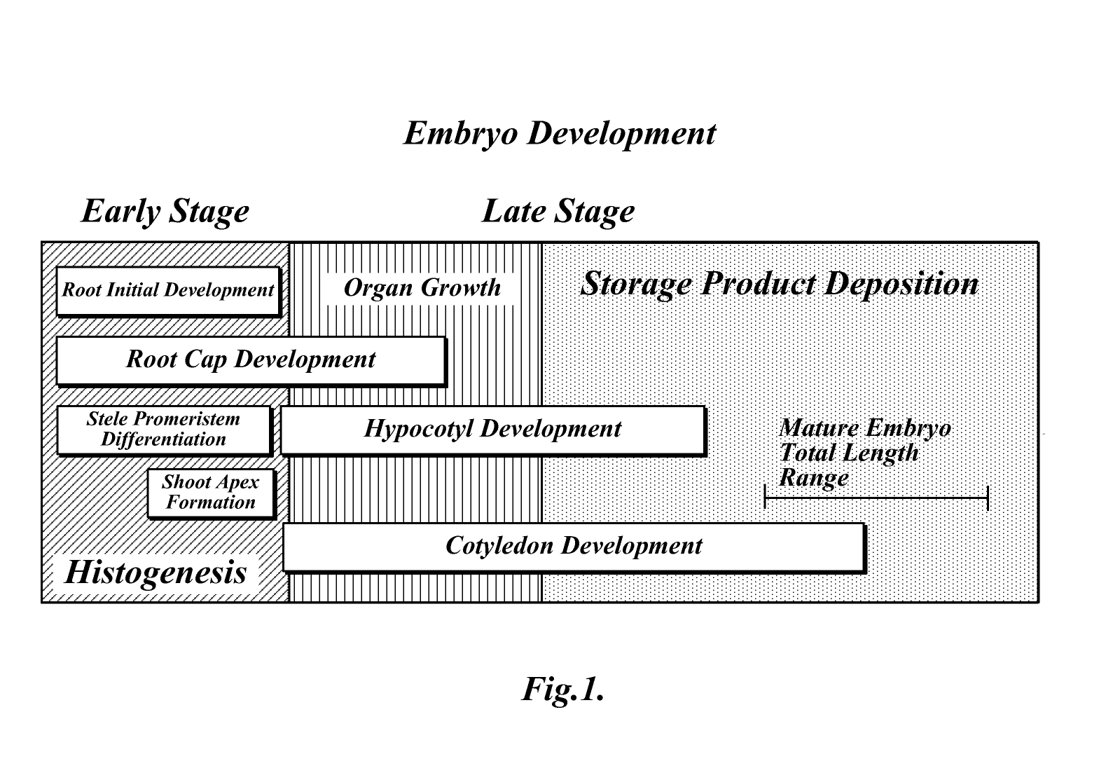

In accordance with the methods of the invention, prior to cold treatment, a first culture of immature embryos is incubated in a first development media, for a first incubation period. As shown in FIG. 1, the development stage of somatic embryos may be divided into the early stage which involves histogenesis (i.e., the formation of different tissues from undifferentiated cells), mid-stage which involves organ growth and the initiation of hypocotyl development and cotyledon development, and the late stage which involves the completion of organ growth, the completion of hypocotyl and cotyledon development (i.e., anatomical maturity) and storage product deposition. In particular, early stage development of an immature embryo includes root initial development, the beginning of root cap development, stele promeristem differentiation, and shoot apex formation. Mid-stage development includes the initiation of hypocotyl development and cotyledon development, and late stage development includes completion of hypocotyl development and cotyledon development, resulting in an anatomically mature embryo.

In accordance with the methods of the invention, immature conifer somatic embryos, such as, for example, pre-cotyledonary conifer somatic embryos, can be prepared from conifer somatic cells, such as cells obtained from conifer embryos. For example, cells from conifer embryos can be induced by hormones to form embryonal suspensor cell masses (ESMs) that can be treated in accordance with the present invention to yield mature conifer somatic embryos. ESMs can be prepared, for example, from pre-cotyledonary embryos removed from seed. For example, the seeds are surface sterilized before removing the pre-cotyledonary embryos, which are then cultured on, or in, an induction medium that permits formation of ESMs which include early stage embryos in the process of multiplication by budding and cleavage. ESMs are typically cultured in a maintenance medium to form pre-cotyledonary somatic embryos. Non-limiting examples of ESM culture conditions and suitable induction and maintenance media are further described below.

In one embodiment of the method of the invention, a first culture comprising immature embryos, such as ESM comprising a plurality of pre-cotyledonary somatic embryos, is cultured in, or on, a first development medium that promotes the development of cotyledonary embryos at a temperature in the range of from 20° C. to 30° C. for a first incubation period prior to cold treatment.

In some embodiments, the first incubation period in development medium is sufficient in length for the formation of at least one of the following structures on a portion (e.g., at least one embryo, at least 10% of the embryos, at least 25%, at least 50%, or at least 75%) of the plurality of embryos in the first embryo culture: one or more embryos with cotyledonary primordia; one or more embryos with cotyledons; one or more embryos with 4+ cotyledons; or one or more embryos with distinct cotyledons with hypocotyl and root regions present.

In one embodiment, the first incubation period is a time period sufficient in length for the formation of one or more cotyledonary primordia on a portion of the plurality of immature conifer somatic embryos incubated in or on the first development media.

The formation of one or more structures on one or more embryos (e.g., cotyledonary primordial or cotyledons) may be determined by visual inspection or imaging analysis of the cultured embryos. Visual inspection or imaging analysis may be optionally carried out under 5-10× magnification.

The length of the first incubation period on development media may be different depending on the genotype. In some embodiments, the first incubation period on development media is from at least six weeks to at least eight weeks in length, such as from seven to eight weeks. The first incubation period on the first development media is carried out at a temperature ranging from 20° C. to 30° C., such as from 20° C. to 25° C. In some embodiments, the first incubation period on the first development media is carried out at a temperature ranging from 20° C. to 22° C. The first incubation period useful for a particular genotype may be determined using the methods described in EXAMPLE 2.

At the end of the first incubation period, for example, when the presence of one or more cotyledonary primordia is observed on a portion of the embryos, or after a time period of at least six weeks, the method comprises exposing a plurality of the immature embryos to a cold temperature in the range of 0° C. to 10° C. for a time period of at least one week up to at least 4 weeks, such as one week, two weeks, three weeks, four weeks, or longer.

In some embodiments, the method further comprises singulating a plurality of individual embryos cultured in, or on, the first development medium. In such embodiments, the embryos may be singulated after the first incubation period, subjected to a cold treatment, and then incubated on a second development media for a second development period. In another embodiment, the embryos may be subjected to a cold treatment, then singulated and incubated on a second development media for a second development period. In some embodiments, the embryos may be subjected to a cold treatment, incubated on a second development media for a second development period, and singulated after development.

Any means of physically separating individual embryos from the first culture of embryos may be used to singulate the embryos in accordance with this embodiment of the methods of the invention. For example, in the context of an embryonal suspensor mass (ESM) culture, physical methods of separation may be used, such as washing away the ESM (e.g., spray singulation via pressure-controlled spray of aqueous liquid), vacuuming away the ESM, vibration, or picking the embryos from the ESM. Other non-limiting examples of useful singulation methods include filtering or sorting embryos based on physical attributes such as size, shape, for example through a sieve, or based on other physical attributes such as surface roughness, hydrophobicity, density or mass.

In some embodiments, the singulation step further comprises picking individual embryos based on one or more selection criteria. For example, visually evaluated screening criteria may be used by a skilled technician or a computerized imaging system to select embryos based on one or more morphological features including, but not limited to, the embryos' size, shape (e.g., axial symmetry), surface texture, color (e.g., no visible greening), absence of split hypocotyls, and no translucent cotyledons. Embryos can also be selected based on criteria relating to chemistry or external structure adsorption, reflectance, transmittance, or emission spectra through the use of near infrared spectroscopy (NIR), as described in U.S. Patent Application Serial No. 2004/0072143 entitled “Methods for Classification of Somatic Embryos,” incorporated herein by reference.

Desirable embryos may be individually picked (via a manual or automated process) out of the first embryo culture (e.g., such as an embryonal suspensor mass), with any suitable instrument, such as tweezers. The embryo picking may be carried out manually or via an automated process, such as described in U.S. Patent Application Serial No. 2004/0267457, entitled “Automated System and Method for Harvesting and Multi-Stage Screening of Plant Embryos,” incorporated herein by reference.

In some embodiments of the method, the picked embryos are laid out directly onto the surface of a second development medium, or onto a porous substrate in contact with a second development medium, which may be in solid or liquid form. In some embodiments, the picked embryos are laid out on a second development medium prior to exposure to a cold treatment. In other embodiments, the embryos are exposed to a cold treatment while on the first development medium, and are then singulated and laid out onto a second development medium.

A porous substrate that is useful in the practice of various embodiments of the methods of the invention typically has a port diameter in the range of from about 5 microns to about 1200 microns, such as from about 50 to 500 microns, such as from about 70 to about 150 microns, such as about 100 microns. The porous material is typically planar and may be any desired shape or dimension chosen for ease of manipulation and for placement in contact with the second development media. Exemplary porous materials include materials that are sterilizable and sufficiently strong to resist tearing when the materials are lifted in order to transfer singulated embryos to subsequent stages of the somatic embryo production process, such as stratification. Examples of useful porous materials include, but are not limited to, membranes, nylon fiber, woven mesh (e.g., nylon, stainless steel or plastic), and polymeric fibers.

### Cold Treatment:

In accordance with the methods of the invention, after the first incubation period of the first development media, the embryos are subjected to a cold treatment. In some embodiments, the cold treatment comprises exposure of the embryos to a temperature in the range of 0° C. to 10° C. (such as 0° C., 1° C., 2° C., 3° C., 4° C., 5° C., 6° C., 7° C., 8° C., 9° C., or 10° C.) for a time period of at least one week up to four weeks, such as for one week, two weeks, three weeks or four weeks. In some embodiments, the embryos are exposed to a temperature in the range of from 0° C. to 5° C. for time period of one to three weeks.

After the embryos have been subjected to a cold treatment, the embryos are incubated on a second development media, or a porous substrate in contact with a second development media, and incubated for a second incubation period on a second development media at a temperature in the range of from 20° C. to 30° C., such as from 20° C. to 25° C. The length of the second incubation period on development media may be different depending on the genotype. In some embodiments, the second incubation period on development media is at least sufficient in length for at least a portion (e.g., at least one embryo, at least 10% of the embryos, at least 25%, at least 50%, more than 50%, or at least 75%) of the plurality of immature conifer somatic embryos to reach anatomical maturity (i.e., possessing developed cotyledons and hypocotyl).

The second incubation period may be different depending on genotype. In some embodiments, the second incubation period is at least one week in length, such as from one week to five weeks in length. In some embodiments, the second incubation period is from two weeks to five weeks in length. In some embodiments, the second incubation period is from two weeks to four weeks in length.

In some embodiments, the embryos are incubated for a total length of time (including first incubation period, cold exposure, and second incubation period) of at least 12 weeks on development media. The length of the second incubation period useful for a particular genotype may be determined using the methods described in EXAMPLE 2. For example, in one exemplary embodiment, the method comprises incubating immature embryos on a first development media at a temperature in the range of 20° C. to 30° C. for 6 weeks, exposing the embryos to a cold temperature in the range of 0° C. to 10° C. for one week, and incubating the embryos on a second development media at a temperature in the range of 20° C. to 30° C. for five weeks. In another exemplary embodiment, the method comprises incubating immature embryos on a first development media at a temperature in the range of 20° C. to 30° C. for 8 weeks, exposing the embryos to a cold temperature in the range of 0° C. to 10° C. for one week, and incubating the embryos on a second development media at a temperature in the range of 20° C. to 30° C. for 3 weeks.

In another exemplary embodiment, the method comprises incubating immature embryos on a first development media at a temperature in the range of 20° C. to 30° C. for 6 weeks, exposing the embryos to a cold temperature in the range of 0° C. to 10° C. for one week, and incubating the embryos on the same development media (i.e., the first development media, without embryo transfer) at a temperature in the range of 20° C. to 30° C. for 5 weeks.

The first and second development media typically contain nutrients that sustain the somatic embryos. Suitable development media typically do not include growth-promoting hormones, such as auxins and cytokinins. In some embodiments, the first and second development media have the same formulation. In some embodiments, the first and second development media have different formulations.

The osmolality of the first and/or second development medium can be adjusted to a value that falls within a desired range, such as from about 250 mM/Kg to about 450 mM/Kg. Typically, an osmolality of 350 mM or higher is advantageous in the methods of the invention. An example of a suitable development medium BM₃ is set forth in EXAMPLE 1 herein. Another example of a suitable development is set forth in EXAMPLES 1 and 2 herein. In some embodiments of the method, the second development medium has a higher osmolality (e.g., from 350 mM/Kg to 450 mM/Kg) than the first development medium (e.g., from 300 mM/Kg to 400 mM/Kg). In some embodiments, the osmolality of the second development media is chosen to match the osmolality of the first development media at the end of the first incubation period.

In some embodiments, the first and/or second development medium comprises PEG at a concentration from 1% to 15%. In some embodiments, the first development medium comprises PEG at a concentration of 7% to 10% (e.g., 7%, 8%, 9%, 10%). In some embodiments, the second development medium comprises PEG at a concentration of 8% to 15% (e.g., 8%, 9%, 10%, 11%, 12%, 13%, 14%, 15%). In some embodiments, the second development medium comprises PEG at a higher concentration than the first development medium.

Maltose may be included in the first and/or second development medium as the principal or sole source of sugar for the somatic embryos. Useful maltose concentrations are within the range of from about 1% to about 2.5%.

The first and/or second development medium may contain gellan gum. Gellan gum is a gelling agent marketed, for example, under the names GELRITE and PHYTAGEL. If gellan gum is included in the development medium, it is typically present at a concentration less than about 0.5%, typically at a concentration from about 0% to about 0.4%. The first and second development media are typically a solid medium, although one or both can be a liquid medium.

The first and/or second development medium may contain an absorbent composition, such as activated charcoal, as described herein, for the induction medium.

In some embodiments, the first and/or second development medium further comprises sucrose and/or abscisic acid. The concentration of abscisic acid in the development medium may be between 0.5 mg/L and 500 mg/L. In some embodiments of the methods of the invention, the concentration of abscisic acid in the development medium is between 1 mg/L and 100 mg/L. In some embodiments, the concentration of abscisic acid in the development medium is between 5 mg/L and 20 mg/L.

In some embodiments of the invention, the first and/or second development medium contains sucrose as the principal or sole source of metabolizable sugar. Useful sucrose concentrations are within the range of about 1% to about 6%.

In some embodiments, after incubation in the second development medium, the embryos are then cultured in, or on, a stratification medium for a period of about one week to about six weeks, at a temperature of from about 1° C. to about 10° C. Typically, the stratification medium is similar or identical to the development medium, but does not contain abscisic acid and has a lower concentration of gellan gum, typically less than about 0.5%. The stratification medium may contain sucrose as the principal or sole source of metabolizable sugar. An exemplary stratification medium is set forth as BM₄ in EXAMPLE 1.

In another aspect, a method is provided for producing mature conifer somatic embryos. The method comprises (a) culturing conifer somatic cells in, or on, an induction medium to yield embryogenic cells; (b) culturing the embryogenic cells prepared in step (a) in, or on, a maintenance medium to multiply the embryogenic cells and form pre-cotyledonary conifer somatic embryos; (c) culturing the pre-cotyledonary conifer somatic embryos formed in step (b) in, or on, a first development medium at a temperature in the range of 20° C. to 30° C. for a first incubation period; (d) exposing the plurality of immature conifer somatic embryos incubated in accordance with step (c) to a cold temperature in the range of 0° C. to 10° C. for a time period of at least one week; and (e) incubating the plurality of immature conifer somatic embryos treated in accordance with step (d) for a second incubation period in, or on, a second development medium at a temperature in the range of 20° C. to 30° C.

Thus, in some embodiments, conifer somatic cells are cultured in, or on, an induction medium to yield embryogenic cells. Embryogenic cells are cells that are capable of producing one or more cotyledonary conifer somatic embryos and include, for example, conifer embryonal suspensor masses. The induction medium typically includes inorganic salts and organic nutrient materials. The osmolality of the induction medium is typically about 160 mg/kg or even lower, but it may be as high as 170 mM/kg. The induction medium typically includes growth hormones. Examples of hormones that can be included in the induction medium are auxins (e.g., 2,4-dichlorophenoxyacetic acid (2,4-D)) and cytokinins (e.g., 6-benzylaminopurine (BAP)). Auxins can be utilized, for example, at a concentration of from 1 mg/L to 200 mg/L. Cytokinins can be utilized, for example, at a concentration of from 0.1 mg/L to 10 mg/L.

The induction medium may contain an absorbent composition, especially when very high levels of growth hormones are used. The absorbent composition can be any composition that is not toxic to the embryogenic cells at the concentrations utilized in the practice of the present methods, and that is capable of absorbing growth-promoting hormones, and toxic compounds produced by the plant cells during embryo development, that are present in the medium. Non-limiting examples of useful absorbent compositions include activated charcoal, soluble poly(vinyl pyrrolidone), insoluble poly(vinyl pyrrolidone), activated alumina, and silica gel. The absorbent composition may be present in an amount, for example, of from about 0.1 g/L to about 5 g/L. An example of an induction medium useful in the practice of the present invention is medium BM₁ set forth in EXAMPLE 1 herein. The induction medium is typically solid, and may be solidified by inclusion of a gelling agent.

Conifer somatic cells are typically cultured in, or on, an induction medium for a period of from three weeks to ten weeks, such as from six weeks to eight weeks, at a temperature of from 10° C. to 30° C., such as from 15° C. to 25° C., or such as from 20° C. to 23° C.

The maintenance medium may be a solid medium, or it may be a liquid medium which can be agitated to promote growth and multiplication of the embryogenic tissue. The osmolality of the maintenance medium is typically higher than the osmolality of the induction medium, typically in the range of 180-400 mM/kg. The maintenance medium may contain nutrients that sustain the embryogenic tissue, and may include hormones, such as one or more auxins and/or cytokinins, that promote cell division and growth of the embryogenic tissue. Typically, the concentrations of hormones in the maintenance medium are lower than their concentration in the induction medium.

It is generally desirable, though not essential, to include maltose as the sole, or principal, metabolizable sugar source in the maintenance medium. Examples of useful maltose concentrations are within the range of from about 1% to about 2.5%. An example of a suitable maintenance medium is medium BM₂ set forth in EXAMPLE 1 herein. Conifer embryogenic cells are typically transferred to fresh maintenance medium once per week.

As described above, pre-cotyledonary conifer somatic cells formed from conifer embryogenic cells are cultured in, or on, a first development medium for a first incubation period, exposed to a cold treatment, and then cultured on a second development medium for a second incubation period. Useful development media and incubation time periods are described supra.

Prior to culturing in the second development media, the cotyledonary somatic embryos can optionally be singulated, as described supra.

After being cultured in the second development media, the cotyledonary somatic embryos can optionally be transferred to a stratification medium, for a further period of culture.

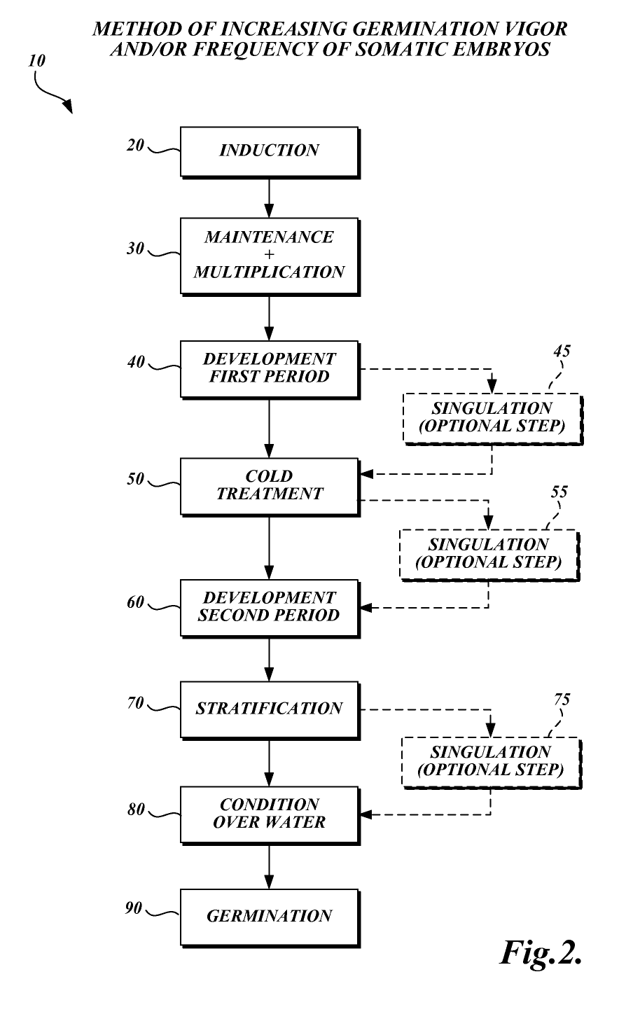

FIG. 2 is a flow chart illustrating various embodiments of the method 10 of increasing germination frequency and/or vigor of somatic embryos in accordance with an aspect of the invention. As shown in FIG. 2, at step 20, a plurality of conifer somatic cells are cultured in, or on, an induction medium to yield embryogenic cells (ESMs). At step 30 ESMs are cultured in a maintenance and multiplication medium to form a plurality of pre-cotyledonary somatic embryos. At step 40, the pre-cotyledonary somatic embryos are cultured in first development media for a first incubation period at a temperature from 20° C. to 30° C. for a period of time sufficient in length for the formation of one or more cotyledonary primordia on a portion of the plurality of immature conifer somatic embryos. At step 50, the embryos are subjected to a cold treatment at a temperature from 0° C. to 10° C. for a period of time from at least one week to four weeks, as described supra. At step 60, the cold-treated embryos are incubated on development media for a second incubation period at a temperature from 20° C. to 30° C. The second development incubation period may be carried out either on the same development media, or on a second development media. As further shown in FIG. 2, the optional step of singulation may be added to the method either before cold treatment (at step 45), or after cold treatment (at step 55) and prior to step 60 (the second incubation on development media), or after stratification (at step 75) and prior to step 80 (conditioning over water). At step 70, the mature embryos are incubated on a stratification medium at a temperature from 0° C. to 10° C. At step 80, the mature embryos are conditioned over water.

As shown in FIG. 2, at step 90, the conifer cotyledonary somatic embryos produced using the methods of the invention can optionally be germinated to form conifer plants which can be grown into coniferous trees, if desired. The cotyledonary embryos may also be disposed within artificial seeds for subsequent germination. The conifer cotyledonary somatic embryos can be germinated, for example, on a solid germination medium, such as the germination medium described in EXAMPLE 1 herein. The germinated plants can then be transferred to soil for further growth. For example, the germinated plants can be planted in soil in a greenhouse and allowed to grow before being transplanted to an outdoor site. Typically, the conifer cotyledonary somatic embryos are illuminated to stimulate germination.

The methods of the invention produce a population of mature conifer somatic embryos with a capacity to germinate at a higher frequency (i.e., produce a higher yield of germinants) than a population of conifer somatic embryos produced according to an otherwise identical method that does not include the step of exposing immature embryos to a cold treatment during development as further described in EXAMPLES 1-2 and shown in FIGS. 3-10, supra.

The following examples merely illustrate the best mode now contemplated for practicing the invention, but should not be construed to limit the invention.

### EXAMPLE 1

This Example describes a method for producing somatic pine embryos from Loblolly Pine using post-development cold treatment (otherwise referred to as stratification), but without exposing the somatic embryos to a cold treatment prior to or during development.

Methods:

Female gametophytes containing zygotic embryos are removed from seeds four to five weeks after fertilization. The seed coats are removed but the embryos are not further dissected out of the surrounding gametophyte other than to excise the nuclear end. The cones are stored at 4° C. until used. Immediately before removal of the immature embryos the seeds are sterilized utilizing an initial washing and detergent treatment followed by a 10 minute sterilization in 15% H₂O₂. The explants are thoroughly washed with sterile distilled water after each treatment.

Tables 1 and 2 set forth exemplary compositions of media useful for producing pine somatic embryos.

TABLE 1
Pinus Taeda Basal Medium (BM)

| Constituent | Concentration (mg/L) |
|---|---|
| NH₄NO₃ | 150.0 |
| KNO₃ | 909.9 |
| KH₂PO₄ | 136.1 |
| Ca(NO₃)₂•4H₂O | 236.2 |
| CaCl₂•4H₂O | 50.0 |
| MgSO₄•7H₂O | 246.5 |
| Mg(NO₃)₂•6H₂O | 256.5 |
| MgCl₂•6H₂O | 50.0 |
| KI | 4.15 |
| H₃BO₃ | 15.5 |
| MnSO₄•H₂O | 10.5 |
| ZnSO₄•7H₂O | 14.4 |
| NaMoO₄•2H₂O | 0.125 |
| CuSO₄•5H₂O | 0.125 |
| CoCl₂•6H₂O | 0.125 |
| FeSO₄•7H₂O | 27.86 |
| Na₂EDTA | 37.36 |
| Maltose | 30,000 |
| myo-Inositol | 200 |
| Casamino acids | 500 |
| L-Glutamine | 1000 |
| Thiamine-HCl | 1.00 |
| Pyridoxine-HCl | 0.50 |
| Nicotinic acid | 0.50 |
| Glycine | 2.00 |
| Gelrite⁺ | 1600 |
| pH adjusted to 5.7 | |

⁺Used if a solid medium is desired.

TABLE 2
Composition of Media for Different Stage Treatments

| | |
|---|---|
| BM₁-Induction | BM + 2,4-D (15 μM) + Kinetin (2 μM) + BAP (2 μM). |
| Medium | |
| BM₂-Maintenance | BM + 2,4-D (5 μM) + Kinetin (0.5 μM) + BAP (0.5 μM). |
| Medium | GELRITE (1600 mg/L) is added when a solid medium is |
| | desired. |
| Dilution Medium | BM + 10 mg/mL abscisic acid + 100-1000 mg/mL additional |
| | myo-inositol, +2.5% Maltose. The following amino acid |
| | mixture is added: L-proline (100 mg/L), L-asparagine |
| | (100 mg/L), L-arginine (50 mg/L), L-alanine (20 mg/L), and |
| | L-serine (20 mg/L). Preferably no maintenance hormones |
| | are present. |
| BM₃- | BM + 25 mg/L abscisic acid + 12% PEG-8000 + 800 mg/L |
| Development | additional myo-inositol + 0.1% activated charcoal + 1% |
| Medium A | glucose, +2.5% Maltose. The following amino acid mixture |
| | is added: L-proline (100 mg/L), L-asparagine (100 mg/L), |
| | L-arginine (50 mg/L), L-alanine (20 mg/L), and L-serine |
| | (20 mg/L). GELRITE (2500 mg/L) is added when a solid |
| | medium is desired. |
| BM₄-Stratification | BM₃ modified by omitting abscisic acid, and PEG-8000. |
| Medium | GELRITE (2500 mg/L) is added when a solid medium is |
| | desired. |
| BM₅-Germination | BM modified by replacing maltose with 2% sucrose. |
| Medium | Myo-inositol is reduced to 100.0 mg/L, glutamine and |
| | casamino acids are reduced to 0.0 mg/L. FeSO₄•7H₂O is |
| | reduced to 13.9 mg/L and Na₂EDTA reduced to 18.6 mg/L. |
| | Agar at 0.8% and activated charcoal at 0.25% are added. |

Induction: Sterile gametophytes with intact embryos are placed on a solid BM₁ culture medium and held in an environment at 20°-25° C. with a 24 hour dark photoperiod for a time of three to five weeks. The length of time depends on the particular genotype being cultured. At the end of this time, a white mucilaginous mass forms in association with the original explants. Microscopic examination typically reveals numerous early stage embryos associated with the mass. These are generally characterized as having a long thin-walled suspensor associated with a small head with dense cytoplasm and large nuclei.

Osmolality of the induction medium may in some instances be as high as 150 mM/kg, and is typically about 120 mM/kg or even lower (such as 110 mM/kg).

Maintenance and Multiplication of Pre-cotyledonary Embryos: Early stage embryos removed from the masses generated in the induction stage are first placed on a BM₂ gelled maintenance and multiplication medium. This differs from the induction medium in that the growth hormones (both auxins and cytokinins) are reduced by at least a full order of magnitude. Osmolality of this medium is at 130 mM/kg or higher (typically within the range of about 120-150 mM/kg for Pinus taeda). The temperature and photoperiod are again 22°-25° C. with 24 hours in the dark. Embryos are cultured 12-14 days on the BM₂ solid medium before transferring to a liquid medium for further subculturing. This liquid medium has the same composition as BM₂, but lacks the gellant. The embryos at the end of the solid maintenance stage are typically similar in appearance to those from the induction stage. After five to six weekly subcultures on the liquid maintenance medium, advanced early stage embryos have formed. These are characterized by smooth embryonal heads, estimated to typically have over 100 individual cells, with multiple suspensors.

Embryo Development

Early stage immature embryos are transferred to a solid development medium. This development medium lacks the growth hormones from maintenance (i.e. kinetin, 2,4-D, BAP). Abscisic acid is typically included to facilitate further development. The further inclusion of an absorbent composition in this medium is advantageous. The absorbent composition may be chosen from a number of chemical materials having high surface area and/or controlled pore size, such as activated charcoal, soluble and insoluble forms of poly(vinyl pyrrolidone), activated alumina, and silica gel. The absorbent composition is normally present at a concentration of about 0.1-5 g/L, more generally about 0.25-2.5 g/L. Gellan gum may be included at a concentration of about 0.25%.

The osmotic potential of this development medium may be raised substantially over that of the maintenance medium. It has been found advantageous to have an osmolality as high as 350 mM/kg or even higher. Development is preferably carried out in complete darkness at a temperature of 20°-25° C. until cotyledonary embryos have developed (e.g., reached anatomical maturity).

Stratification: After 7 to 12 weeks on development medium, in accordance with conventional methods, cotyledonary embryos are transferred to stratification medium BM₄. This medium is similar to development medium but lacks abscisic acid, PEG-8000, and gellan gum. Embryos are cultivated on stratification medium at between about 1° C. and about 10° C. in the dark for between three to six weeks. In accordance with convention methods, embryos are singulated either after development or after stratification.

Drying: The mature embryos still on their filter paper support are lifted from the pad and placed in a closed container over H₂O at a relative humidity of 97%, for a period of about two to three weeks.

Germination: The dried mature embryos are rehydrated by placing them, while still on the filter paper support, for about 24 hours on a pad saturated with liquid germination medium. The embryos are then placed individually on solid BM₅ medium for germination. This is a basal medium lacking growth hormones which is modified by reducing sucrose, myo-inositol and organic nitrogen. The embryos are incubated on BM₅ medium for about ten weeks under environmental conditions of 20°-25° C., and a 16-hour light-8-hour dark photoperiod, until the resulting plantlets have a well developed radicle and hypocotyl and green cotyledonary structure and epicotyl.

Because of the reduced carbohydrate concentration, the osmotic potential of the germination medium may be further reduced below that of the development medium. It is normally below about 150 mM/kg (such as about 100 mM/kg).

### EXAMPLE 2

This Example demonstrates the improvement in germination frequency and vigor observed when immature embryos were subjected to a cold treatment during embryo development.

Rationale: This experiment was carried out to evaluate whether a cold treatment during development would improve the germination frequency and/or vigor of germinants of somatic pine embryos. Somatic pine embryos were incubated in development media at 20° C. to 25° C. for a first incubation period, subjected to a cold treatment, then incubated for a second development period followed by either stratification or no stratification. The effect of singulation during development (either prior to or after cold treatment) was also tested.

Methods:

Induction and Maintenance of Pre-Cotyledonary Embryos:

Somatic embryos from four different Loblolly Pine genotypes A, B, C and D were induced as described in Example 1 and were maintained in maintenance media M2: (BM medium TABLE 2+1.1 mg/l 2,4-D; 0.1 mg/ml 6-BAP, 0.1 mg/ml kinetin, and 1 mg/ml ABA).

Development of Pre-Cotyledonary Embryos:

The experiment was blocked by treatments, with five blocks for each genotype (A, B, C, and D). For each block, 12 plates of each genotype were plated onto plates containing 100 ml semi-solid development media A (dev A), described above in TABLE 2.

To each plate, individual 10 micron nylon mesh squares (1.5 inches×1.5 inches) were added to permit undisturbed removal of the entire culture when desired.

Conifer somatic embryo cells of each genotype (A, B, C and D) that were grown in liquid maintenance medium were allowed to settle. The settled cell volume (SCV) was measured in either a flask or a Falcon tube by drawing a line on the flask or tube. Using a fritted glass pipette and hose with hand valve, the medium was completely aspirated, leaving only the cells in the flask/vessel. Rinse medium was added up to the marked line and mixed with the cells. 60 plates (12 plates×5 blocks) were plated per genotype, with 0.5 mls of SCV mixed with 0.5 ml rinse media plated on each plate.

Mid-Development Assessment and Cold Treatment

Following 6 weeks of incubation on development media A at 20° C. to 22° C., the cotyledonary development of the embryos of each genotype (A, B, C and D) was assessed to determine if there was formation of one or more cotyledonary primordia on a portion of the plurality of immature conifer somatic embryos. For the embryo cultures that met this criteria (genotypes A and C), the plated embryos were subjected to a cold treatment at either 6 or 8 weeks. For the embryo cultures that did not meet this criteria (genotypes B and D) the embryos were incubated in the development media for an additional 2 weeks and reassessed prior to cold treatment. As shown in TABLE 3, at 6 weeks after incubation on the first development media, twenty treatment plates were moved to 5° C. for two weeks. At 8 weeks after incubation on the first development media another twenty treatment plates were moved to 5° C. for two weeks.

Either prior to or after the cold treatment, a subset of the treatment plates were singulated to fresh development media prior to incubation for a second development period lasting from 2 to 4 weeks.

At the end of the second development period, a subset of plates were subjected to stratification on stratification medium BM₄ at 5° C. for four weeks. Another subset of plates was moved directly to conditioning over water for a period of two weeks, and was not subjected to stratification. Depending on the treatment condition shown in TABLE 3, the conditioning over water was carried out by moving the mature embryos (still on the nylon support), or singulating the mature embryos off of the development media onto filter paper and placing them in a closed container over H₂0 at a relative humidity of 97%.

Germination: The mature embryos were rehydrated by placing them, while still on the filter paper, for about 3 hours on solid germination medium BM₅. This is a basal medium lacking growth hormones which is modified by reducing sucrose, myo-inositol and organic nitrogen. The embryos were incubated on BM₅ medium for about six weeks under environmental conditions of 20°-22° C., and a 16-hour light-8-hour dark photoperiod, until the resulting plantlets had a well developed radicle and hypocotyl and green cotyledonary structure and epicotyl.

A summary of the twelve different treatment conditions is provided below in TABLE 3. As shown in TABLE 3, the factors tested were Stratification (yes or no), Singulation (8 weeks into development, or after development and before conditioning over water (COW)), and Cold Shock (none, 6 weeks or 8 weeks into development). Plates were the experimental units.

As shown in TABLE 3, for treatment groups 1, 2, 3 and 7, 8, 9, the embryos were singulated at 6 or 8 weeks, while for treatment groups 4, 5, 6 and 10, 11 and 12 the embryos were singulated after development. For treatment groups 1-6 the embryos did not undergo stratification, while the embryos in treatment groups 7-12 did undergo stratification. Finally, for treatment groups 1, 4, 7 and 10 the embryos were exposed to a cold shock at 6 weeks into development; for treatment groups 2, 5, 8 and 11 the embryos were exposed to a cold shock at 8 weeks into development and embryos in treatment groups 3, 6, 9 and 12 were not exposed to a cold shock during development.

TABLE 3
Treatment Conditions

| | | | Cold | | | | | | Germination (1 + 2) |
|---|---|---|---|---|---|---|---|---|---|
| | Develop- | | shock | | | | | | Fraction from |
| Treatment | ment | | (2 | | Development | Stratification | | Condition | Model 2 (mean of |
| Group | period 1 | Singulation | weeks) | Singulation | period 2 | (4 weeks) | Singulation | over water | 2 genotypes) |
| 1 | 6 weeks | − | + | + | 4 weeks | − | − | + | 0.308 |
| 2 | 8 weeks | + | + | − | 2 weeks | − | − | + | 0.228 |
| 3 | 8 weeks | − | − | + | 4 weeks | − | − | + | 0.343 |
| 4 | 6 weeks | − | + | − | 4 weeks | − | + | + | 0.151 |
| 5 | 8 weeks | − | + | − | 2 weeks | − | + | + | 0.123 |
| 6 | 12 weeks | − | − | − | − | − | + | + | 0.178 |
| 7 | 6 weeks | − | + | + | 4 weeks | + | − | + | 0.143 |
| 8 | 8 weeks | + | + | − | 2 weeks | + | − | + | 0.259 |
| 9 | 8 weeks | − | − | + | 4 weeks | + | − | + | 0.142 |
| 10 | 6 weeks | − | + | − | 4 weeks | + | + | + | 0.552 |
| 11 | 8 weeks | − | + | − | 2 weeks | + | + | + | 0.594 |
| 12 | 12 weeks | − | − | − | − | + | + | + | 0.480 |

Results:

After 6 weeks incubation on germination medium, the embryos were assessed for germination rate (fraction of the total embryos incubated on germination media), root length and hypocotyl length.

A category 1 germinant included the following features: the presence of at least a 1 mm root (no nubbins), the presence of at least 5 epicotyl leaves with a minimum length of 5 mm, no large scale hypocotyl ruptures, and the hypocotyl not bent greater than 90 degrees.

A category 1+2 germinant included the same features as a category 1 germinant described above, but without the minimum requirement for the number of epicotyl leaves and without the minimum requirement for length of epicotyl leaves.

Germination rate was analyzed with a generalized linear model using a logit link with SAS Proc Genmod. Root length and hypocotyl length were analyzed with linear mixed model using SAS Proc Mixed. Mean comparisons were made using Tukey's multiple comparison procedure with α=0.10. Root length was first transformed by taking the natural log to stabilize its variance.

Model 1 refers to a smaller set of treatments, excluding the treatments involving the cold shock at 6 weeks for all genotypes A, B, C and D.

Model 2 refers to the full set of 12 treatments analyzed for genotypes A and C.

Category 1+2 Germination Frequency Analysis:

FIG. 3A graphically illustrates the germination fraction (category 1+2) for genotype A somatic pine embryos that were or were not exposed to a cold treatment at various time points during development.

FIG. 3B graphically illustrates the germination fraction (category 1+2) for genotype B somatic pine embryos that were or were not exposed to a cold treatment at various time points during development.

FIG. 3C graphically illustrates the germination fraction (category 1+2) for genotype C somatic pine embryos that were or were not exposed to a cold treatment at various time points during development.

FIG. 3D graphically illustrates the germination fraction (category 1+2) for genotype D somatic pine embryos that were or were not exposed to a cold treatment at various time points during development.

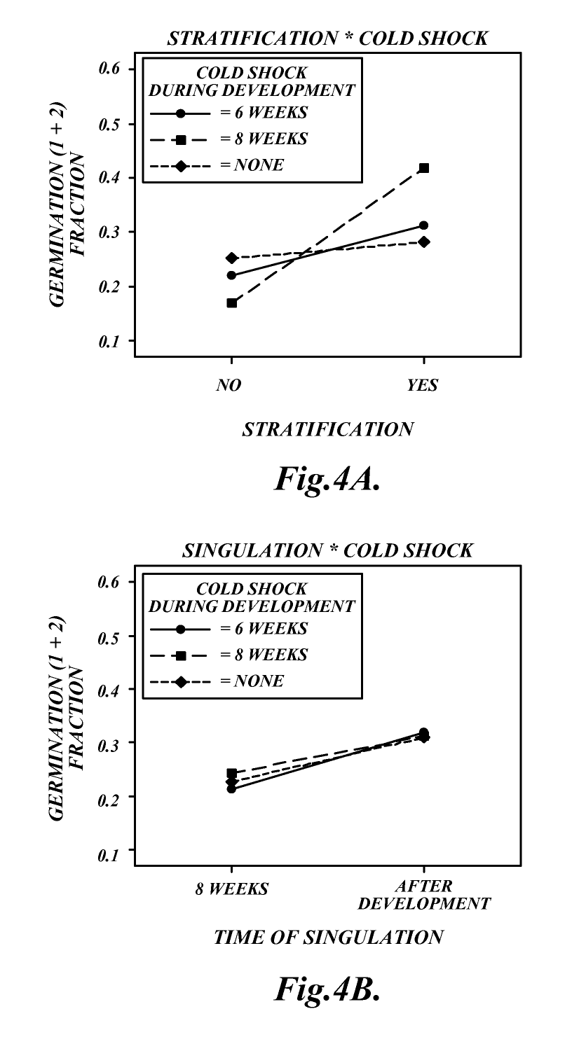

The treatment factor results shown in FIGS. 3A to 3D are summarized below in TABLE 4. As shown in FIG. 4A, the combination of cold shock treatment during development and stratification raised germination frequency from 27% to 42% for several genotypes.

TABLE 4
P-Values for treatment factor effects on Germination (1 + 2) for Model
1 (8 treatments, 4 genotypes) and Model 2 (12 treatments, 2 genotypes)

| Treatment | Model 1 P-value | Model 2 P-value |
|---|---|---|
| Stratification (Y, N) | <0.0001 | <0.0001 |
| Singulation (8 wk, after | 0.082 | 0.002 |
| development) | | |
| Cold Shock (none, 6 weeks into | 0.74 | 0.92 |
| development, 8 weeks into | | |
| development) | | |
| Stratification*Singulation | <0.0001 | <0.0001 |
| Stratification*Cold Shock | 0.005 | 0.003 |
| Singulation*Cold Shock | 0.34 | 0.84 |
| Stratification*Singulation*Cold | 0.42 | 0.56 |
| Shock | | |

The combination of cold shock during development with stratification was found to significantly improve germination frequency, as illustrated in FIG. 4A. As shown in FIG. 4A, the highest germination fraction (category 1+2 germinants) was observed for embryos treated with a cold shock treatment at 8 weeks into development followed by a second development period of 2 weeks and stratification.

As further shown in TABLE 4, the combination of cold shock during development with singulation after the second development period was found to significantly improve germination frequency, as illustrated in FIG. 4B.

TABLE 5 compares the germination fraction (category 1+2 germinants) for Model 2, demonstrating that the effect of cold shock treatment is dependent on the stratification treatment.

TABLE 5
Germination fractions for Model 2 (12 treatments, 2 genotypes)
Lsmeans and 90% Confidence Limits for Germination (1 + 2).

| | Germination | | |
|---|---|---|---|
| Treatment | Fraction (1 + 2) | L90 | U90 |
| 1 | 0.308 | 0.240 | 0.384 |
| 2 | 0.228 | 0.168 | 0.302 |
| 3 | 0.343 | 0.272 | 0.423 |
| 4 | 0.151 | 0.103 | 0.215 |
| 5 | 0.123 | 0.078 | 0.187 |
| 6 | 0.178 | 0.127 | 0.244 |
| 7 | 0.143 | 0.09.1 | 0.217 |
| 8 | 0.259 | 0.196 | 0.334 |
| 9 | 0.142 | 0.094 | 0.208 |
| 10 | 0.552 | 0.474 | 0.627 |
| 11 | 0.594 | 0.515 | 0.668 |
| 12 | 0.480 | 0.404 | 0.558 |

TABLE 6, below, provides a comparison of the germination fraction (category 1+2 germinants) for embryos singulated at 8 weeks, followed by stratification, combined with no cold shock treatment during development, or cold shock at 6 or 8 weeks into development.

TABLE 6
Comparison of Cold Shock Treatments for Germination (1 + 2)
in combination with Stratification and 8 week Singulation

| | Germination | Test at α = | | |
|---|---|---|---|---|
| Cold Shock | (1 + 2) Fraction | 0.10 | L90 | U90 |
| None | 0.142 | a | 0.094 | 0.208 |
| 6 weeks into | 0.143 | a | 0.091 | 0.217 |
| development | | | | |
| 8 weeks into | 0.259 | b | 0.196 | 0.334 |
| development | | | | |

Estimates in Table 6 are from Model 2. L90 and U90 are the lower and upper 90% confidence limits, respectively, for each mean. The column labeled “Test at α=0.10” summarizes test results comparing combined means. Means with the same symbol are not statistically different at α=0.10.

Category 1 Germination Frequency Analysis:

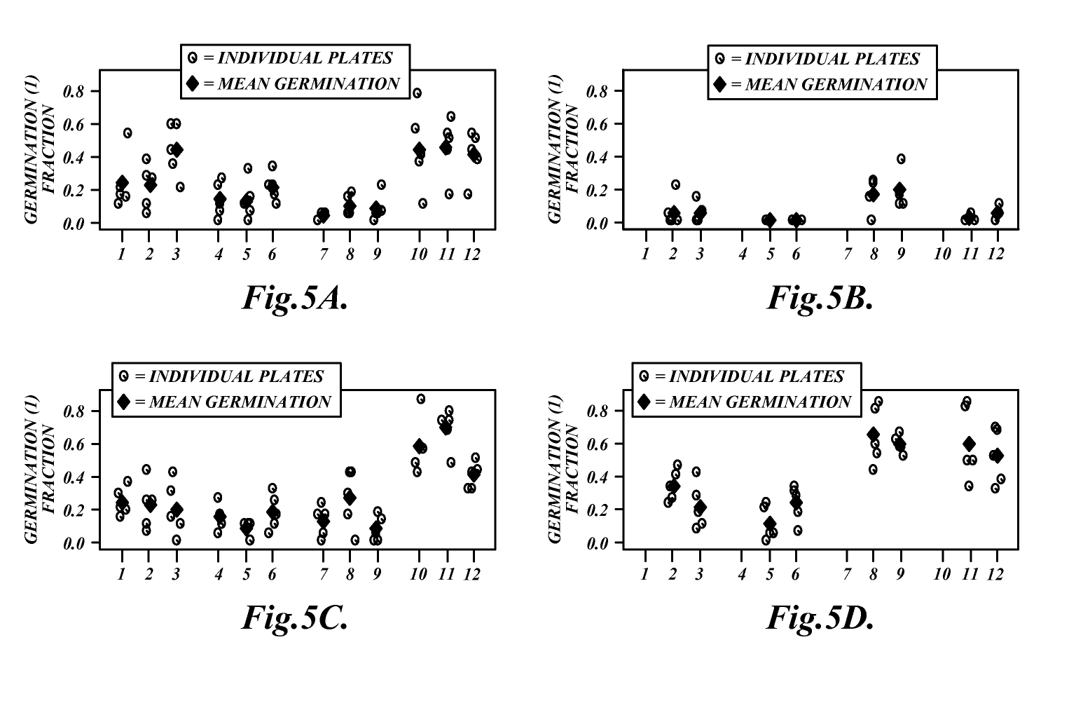

FIG. 5A graphically illustrates the germination fraction (category 1) for genotype A somatic pine embryos that were or were not exposed to a cold treatment at various time points during development.

FIG. 5B graphically illustrates the germination fraction (category 1) for genotype B somatic pine embryos that were or were not exposed to a cold treatment at various time points during development.

FIG. 5C graphically illustrates the germination fraction (category 1) for genotype C somatic pine embryos that were or were not exposed to a cold treatment at various time points during development.

FIG. 5D graphically illustrates the germination fraction (category 1) for genotype D somatic pine embryos that were or were not exposed to a cold treatment at various time points during development.

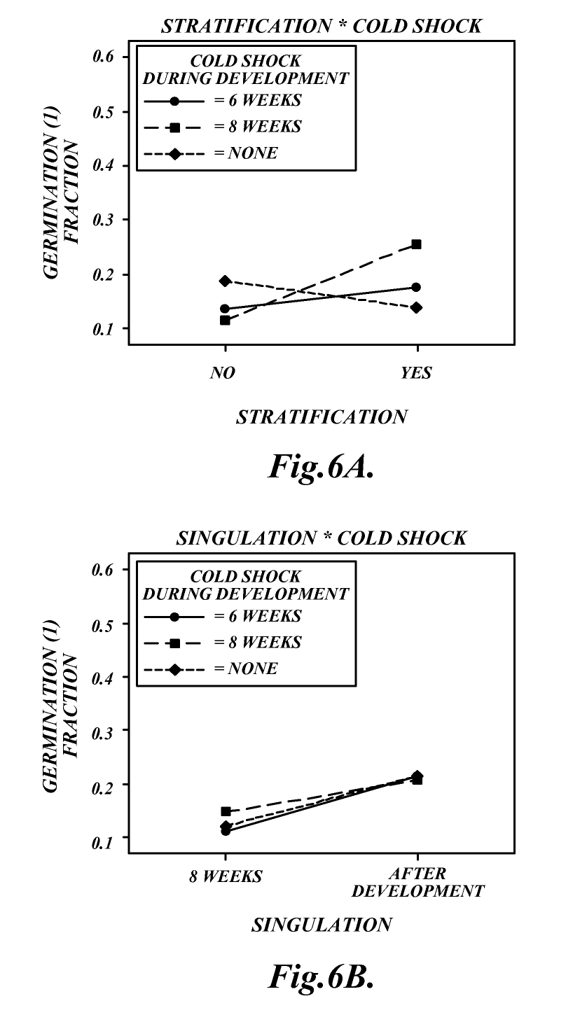

The treatment factor results shown in FIGS. 5A to 5D are summarized below in TABLE 7. As shown in FIG. 6A, the combination of cold shock treatment during development and stratification raised germination frequency from 15% to 25% for several genotypes.

TABLE 7
P-Values for treatment factor effects on Germination (1) for Model 1
(8 treatments, 4 genotypes) and Model 2 (12 treatments, 2 genotypes)

| | | Model 2 |
|---|---|---|
| Treatment | Model 1 P Value | P Value |
| Stratification (Y, N) | <0.0001 | 0.057 |
| Singulation (8 wk, after development) | 0.65 | <0.0001 |
| Cold Shock (none, 6 weeks into | 0.58 | 0.77 |
| development, 8 weeks into development) | | |
| Stratification*Singulation | <0.0001 | <0.0001 |
| Stratification*Cold Shock | 0.01 | 0.002 |
| Singulation*Cold Shock | 0.52 | 0.61 |
| Stratification*Singulation*Cold Shock | 0.25 | 0.88 |

The combination of cold shock during development with stratification was found to significantly improve germination frequency, as illustrated in FIG. 6A. As shown in FIG. 6A, the highest germination frequency (category 1 germinants) was observed for embryos treated with a cold shock treatment at 8 weeks into development followed by a second development period of 2 weeks and stratification.

The combination of cold shock during development with singulation after the second development period was found to significantly improve germination frequency, as illustrated in FIG. 6B.

TABLE 8 compares the germination fraction (category 1 germinants) for Model 2, demonstrating that the effect of cold shock treatment is dependent on the stratification treatment.

TABLE 8
Germination fractions for Model 2 (12 treatments, 2 genotypes)
Lsmeans and 90% Confidence Limits for Germination (1).

| | Germination Fraction | | |
|---|---|---|---|
| Treatment | (category 1 germinants) | L90 | U90 |
| 1 | 0.177 | 0.129 | 0.238 |
| 2 | 0.161 | 0.114 | 0.222 |
| 3 | 0.237 | 0.180 | 0.305 |
| 4 | 0.103 | 0.067 | 0.154 |
| 5 | 0.079 | 0.047 | 0.131 |
| 6 | 0.143 | 0.100 | 0.199 |
| 7 | 0.067 | 0.036 | 0.120 |
| 8 | 0.131 | 0.089 | 0.187 |
| 9 | 0.056 | 0.030 | 0.101 |
| 10 | 0.384 | 0.318 | 0.456 |
| 11 | 0.430 | 0.361 | 0.503 |
| 12 | 0.301 | 0.240 | 0.370 |

TABLE 9 below, provides a comparison of the germination fraction (category 1 germinants) for embryos singulated at 8 weeks, followed by stratification, combined with no cold shock treatment during development, or cold shock at 6 or 8 weeks into development.

TABLE 9
Comparison of Germination (category 1 germinants)
fraction for embryos treated with Cold Shock,
Stratification and 8 week Singulation.

| | Germination (1) | Test at α = | | |
|---|---|---|---|---|
| Cold Shock | Fraction | 0.10 | L90 | U90 |
| None | 0.056 | a | 0.030 | 0.101 |
| 6 weeks into | 0.067 | ab | 0.036 | 0.120 |
| development | | | | |
| 8 weeks into | 0.131 | b | 0.089 | 0.187 |
| development | | | | |

Estimates in Table 9 are from Model 2. L90 and U90 are the lower and upper 90% confidence limits, respectively, for each mean. The column labeled “Test at α=0.10” summarizes test results comparing combined means. Means with the same symbol are not statistically different at α=0.10.

Root Length Analysis:

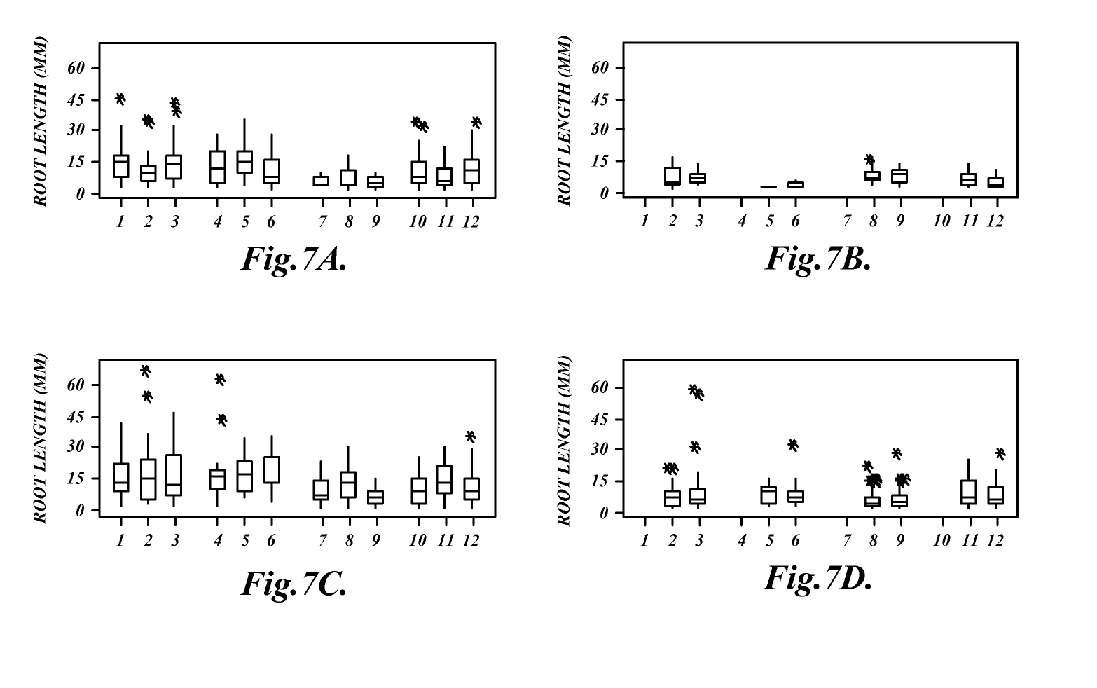

FIGS. 7A-7D are box plots graphically illustrating the distribution of root length (mm) of the germinants (category 1+2) resulting from the embryos treated according to treatments 1 to 12 as described in TABLE 3. The box shows the middle 50% of the data, and the line in the box is the median. The lines extending from the boxes show the range of the rest of the data, apart from the outliers. The symbol “*” indicates outliers.

FIG. 7A graphically illustrates the distribution of root length (mm) for germinants of genotype A resulting from somatic embryos treated with or without a cold treatment during development.

FIG. 7B graphically illustrates the distribution of root length (mm) for germinants of genotype B resulting from somatic embryos treated with or without a cold treatment during development.

FIG. 7C graphically illustrates the distribution of root length (mm) for germinants of genotype C resulting from somatic embryos treated with or without a cold treatment during development.

FIG. 7D graphically illustrates the distribution of root length (mm) for germinants of genotype D resulting from somatic embryos treated with or without a cold treatment during development.

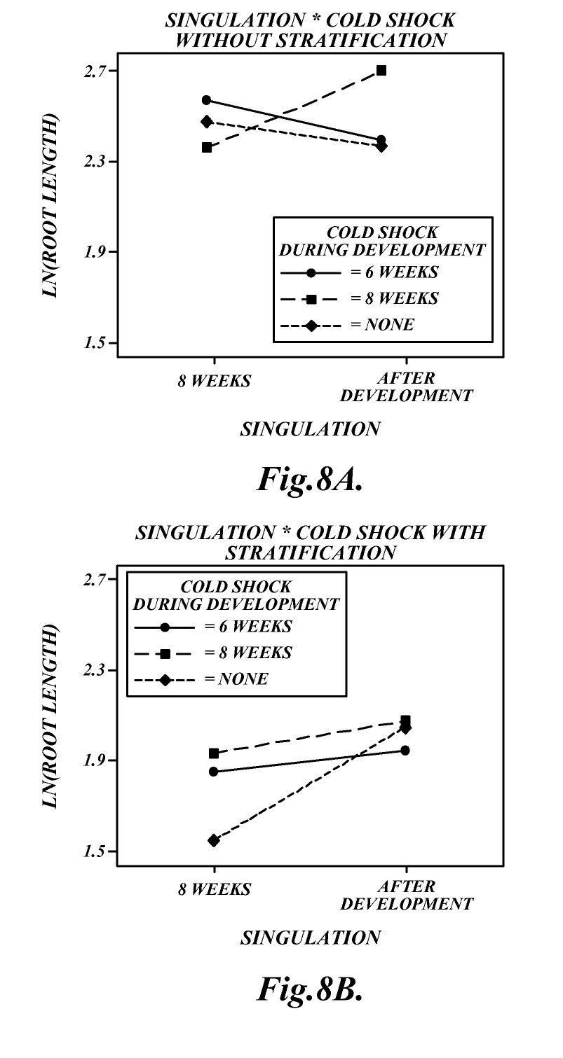

The treatment factor results shown in FIGS. 7A to 7D are summarized below in TABLE 10. As shown below in FIG. 8B, the combination of cold shock treatment during development, singulation at 8 weeks into development, and stratification increases mean root length of the resulting germinants.

TABLE 10
P-Values for treatment factor effects on Root Length for Model 1
(8 treatments, 4 genotypes) and Model 2 (12 treatments, 2 genotypes)

| Treatment | Model 1 P Value | Model 2 P Value |
|---|---|---|
| Stratification (Y, N) | <0.0001 | <0.0001 |
| Singulation (8 wk, after | 0.009 | 0.044 |
| development) | | |
| Cold Shock (none, 6 weeks | 0.39 | 0.14 |
| into development, 8 weeks | | |
| into development) | | |
| Stratification*Singulation | 0.50 | 0.09 |
| Stratification*Cold Shock | 0.78 | 0.82 |
| Singulation*Cold Shock | 0.03 | 0.18 |
| Stratification*Singulation* | 0.09 | 0.04 |
| Cold Shock | | |

The combination of cold shock at 8 weeks during development with singulation after development was found to significantly improve root length of germinants, as illustrated in FIG. 8A. As shown in FIG. 8A, the longest root length of category 1+2 germinants was observed for embryos that were singulated after development and subjected to a cold shock treatment at eight weeks into development.

As further shown in TABLE 10, the combination of cold shock during development with singulation and stratification was found to significantly improve root length of category 1+2 germinants, as illustrated in FIG. 8B. As shown in FIG. 8B, the longest root length of category 1+2 germinants was observed for embryos treated with singulation, a cold shock treatment at 8 weeks into development and stratification.

TABLE 11 compares the root length (category 1+2 germinants) for Model 2, demonstrating that the effect of cold shock treatment on root length is dependent on the singulation and stratification treatment.

TABLE 11
Root Length for Model 2 (12 treatments, 2 genotypes)
Lsmeans and 90% Confidence Limits. All values have
been transformed to the natural scale.

| Treatment | Root Length (mm) | Stderr | L90 | U90 |
|---|---|---|---|---|
| 1 | 13.00 | 1.23 | 9.26 | 18.25 |
| 2 | 10.56 | 1.24 | 7.42 | 15.03 |
| 3 | 11.83 | 1.23 | 8.44 | 16.58 |
| 4 | 10.91 | 1.25 | 7.52 | 15.83 |
| 5 | 14.81 | 1.27 | 9.98 | 21.99 |
| 6 | 10.65 | 1.25 | 7.42 | 15.27 |
| 7 | 6.30 | 1.27 | 4.24 | 9.36 |
| 8 | 6.87 | 1.23 | 4.86 | 9.72 |
| 9 | 4.68 | 1.26 | 3.19 | 6.85 |
| 10 | 6.94 | 1.22 | 5.02 | 9.58 |
| 11 | 7.90 | 1.22 | 5.72 | 10.90 |
| 12 | 7.69 | 1.22 | 5.55 | 10.65 |

TABLE 12, below, provides a comparison of the root length for category 1+2 germinants obtained from embryos singulated at 8 weeks, followed by stratification, in combination with either no cold shock treatment during development, or cold shock at 6 or 8 weeks into development

TABLE 12
Comparison of Root Length in embryos treated with
Cold Shock, Stratification with 8 week Singulation.

| | Root Length | Test at α = | | |
|---|---|---|---|---|
| Cold Shock | (mm) | 0.10 | L90 | U90 |
| None | 4.68 | a | 3.19 | 6.85 |
| 6 weeks into | 6.30 | ab | 4.24 | 9.36 |
| development | | | | |
| 8 weeks into | 6.87 | b | 4.86 | 9.72 |
| development | | | | |

Estimates in Table 12 are from Model 2. L90 and U90 are the lower and upper 90% confidence limits, respectively, for each mean. The column labeled “Test at α=0.10” summarizes test results comparing combined means. Means with the same symbol are not statistically different at α=0.10.

Hypocotyl Length Analysis:

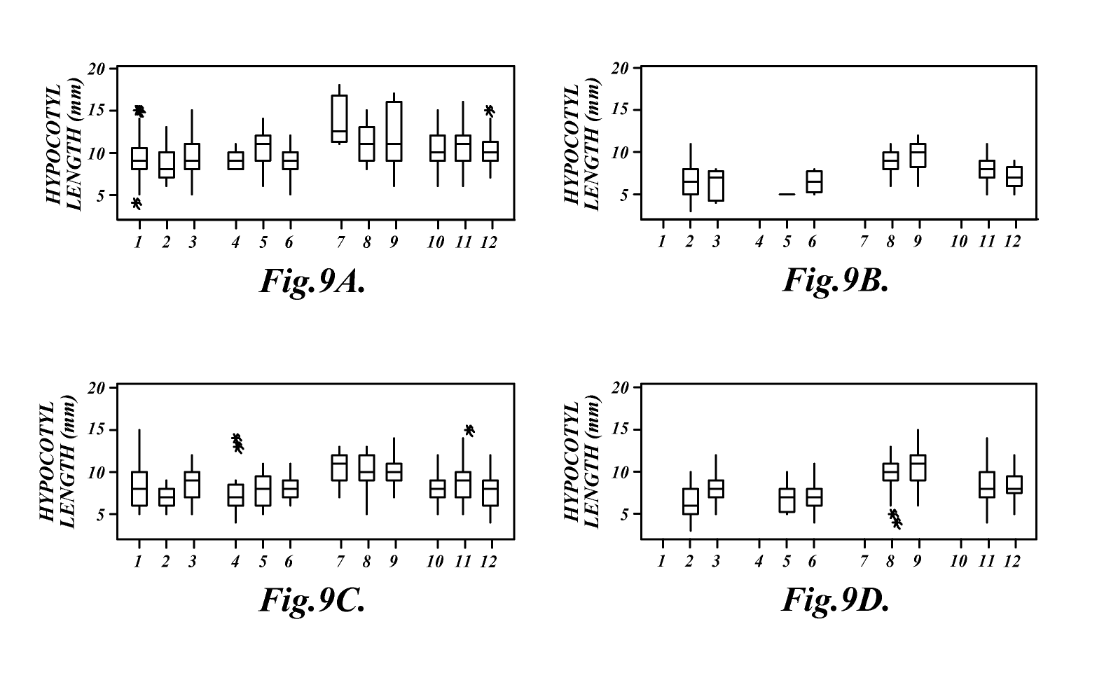

FIGS. 9A-9D are box plots graphically illustrating the distribution of hypocotyl length (mm) of the germinants (category 1+2) resulting from the embryos treated according to treatments 1 to 12 as described in TABLE 3. The box shows the middle 50% of the data, and the line in the box is the median. The lines extending from the boxes show the range of the rest of the data, apart from the outliers. The symbol “*” indicates outliers.

FIG. 9A graphically illustrates the distribution of hypocotyl length (mm) for germinants of genotype A resulting from somatic embryos treated with or without a cold treatment during development.

FIG. 9B graphically illustrates the distribution of hypocotyl length (mm) for germinants of genotype B resulting from somatic embryos treated with or without a cold treatment during development.

FIG. 9C graphically illustrates the distribution of hypocotyl length (mm) for germinants of genotype C resulting from somatic embryos treated with or without a cold treatment during development.

FIG. 9D graphically illustrates the distribution of hypocotyl length (mm) for germinants of genotype D resulting from somatic embryos treated with or without a cold treatment during development.

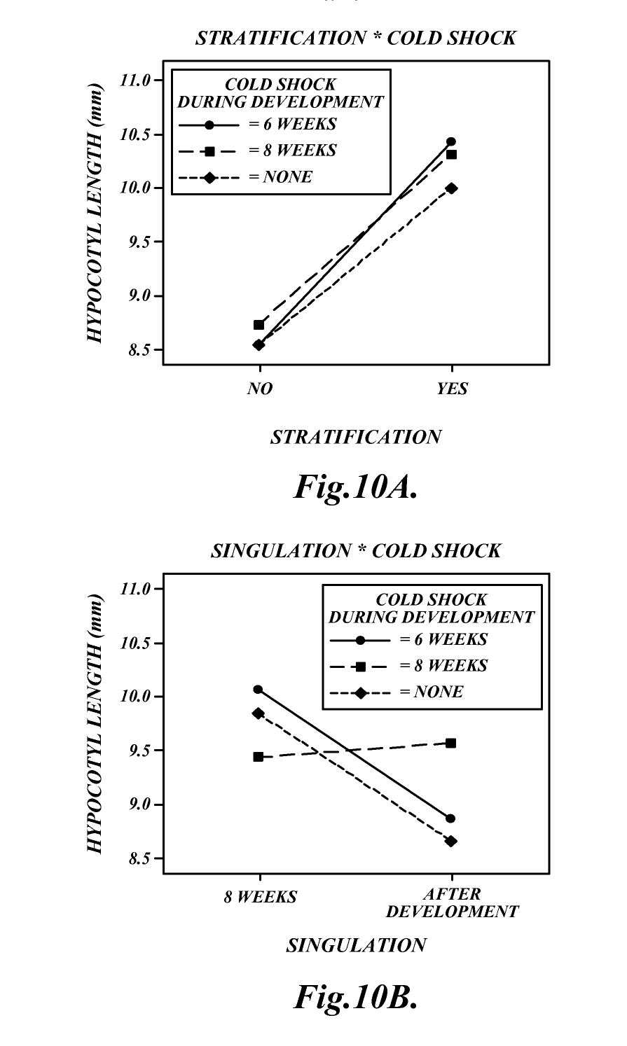

The treatment factor results shown in FIGS. 9A to 9D are summarized below in TABLE 13. As shown below in FIG. 10B, the combination of cold shock treatment during development and singulation at 8 weeks into development increased the mean hypocotyl length of the resulting germinants. It was also observed that the combination of cold shock treatment during development and stratification increased the mean hypocotyl length of the resulting germinants.

TABLE 13
P-Values for treatment factor effects on Hypocotyl Length for Model
1 (8 treatments, 4 genotypes) and Model 2 (12 treatments, 2 genotypes)

| | Model 1 P | Model 2 P |
|---|---|---|
| Treatment | Value | Value |
| Stratification (Y, N) | <0.0001 | <0.0001 |
| Singulation (8 weeks, after | <0.0001 | <0.0001 |
| development) | | |
| Cold Shock (none, 6 weeks into | 0.38 | 0.39 |
| development, 8 weeks into | | |
| development) | | |
| Stratification*Singulation | <0.0001 | <0.0001 |
| Stratification*Cold Shock | 0.66 | 0.54 |
| Singulation*Cold Shock | <0.0001 | 0.0005 |
| Stratification*Singulation*Cold | 0.67 | 0.50 |
| Shock | | |

The combination of cold shock at 6 weeks during development with stratification was found to improve hypocotyl length of germinants, as illustrated in FIG. 10A. As shown in FIG. 10A, the longest hypocotyl length of category 1+2 germinants was observed for embryos treated with a cold shock treatment at 6 weeks into development followed by stratification.

The combination of cold shock at 6 weeks during development with singulation at 8 weeks into development was also found to improve hypocotyl length of category 1+2 germinants, as illustrated in FIG. 10B. As shown in FIG. 10B, the longest hypocotyl length of category 1+2 germinants was observed for embryos treated with a cold shock treatment at 6 weeks into development, with singulation at 8 weeks into development.

TABLE 14 compares the hypocotyl length (category 1+2 germinants) for Model 2, demonstrating that the effect of cold shock treatment on hypocotyl length is dependent on the singulation and/or stratification treatment.

TABLE 14
Hypocotyl Length for Model 2 (12 treatments, 2 genotypes)
Lsmeans and 90% Confidence Limits.

| | Hypocotyl | | | |
|---|---|---|---|---|
| Treatment | Length (mm) | Stderr | L90 | U90 |
| 1 | 8.56 | 0.87 | 7.12 | 10.00 |
| 2 | 8.04 | 0.89 | 6.58 | 9.50 |
| 3 | 8.73 | 0.87 | 7.30 | 10.17 |
| 4 | 8.49 | 0.90 | 7.00 | 9.98 |
| 5 | 9.39 | 0.93 | 7.86 | 10.91 |
| 6 | 8.33 | 0.89 | 6.86 | 9.80 |
| 7 | 11.55 | 0.93 | 10.03 | 13.08 |
| 8 | 10.82 | 0.88 | 9.37 | 12.27 |
| 9 | 10.95 | 0.91 | 9.44 | 12.45 |
| 10 | 9.22 | 0.86 | 7.81 | 10.64 |
| 11 | 9.74 | 0.86 | 8.32 | 11.15 |
| 12 | 8.98 | 0.86 | 7.56 | 10.40 |

TABLE 15, below, provides a comparison of the hypocotyl length for category 1+2 germinants obtained from embryos singulated at 8 weeks, followed by stratification, in combination with either no cold shock treatment during development, or cold shock at 6 or 8 weeks into development

TABLE 15
Comparison of Hypocotyl Length in embryos treated with
Cold Shock, Stratification with 8 week Singulation.

| | Hypocotyl | Test at | | |
|---|---|---|---|---|
| Cold Shock | Length (mm) | α = 0.10 | L90 | U90 |
| None | 10.95 | a | 9.44 | 12.45 |
| 6 weeks into | 11.55 | a | 10.03 | 13.08 |
| development | | | | |
| 8 weeks into | 10.82 | a | 9.37 | 12.27 |
| development | | | | |

Estimates in Table 15 are from Model 2. L90 and U90 are the lower and upper 90% confidence limits, respectively, for each mean. The column labeled “Test at α=0.10” summarizes test results comparing combined means. Means with the same symbol are not statistically different at α=0.10.

Overall Conclusion: The results from this experiment demonstrate that exposing immature somatic pine embryos to a cold shock at a time between 8 and 10 weeks into the development stage was effective to raise germination frequency from 27% up to 42% for category 1+2 germinants for several genotypes, and was effective to raise germination frequency from 15% to 25% for category 1 germinants for several genotypes. In some cases, the combination of a cold shock treatment during development followed by stratification resulted in improved germination frequency.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method of increasing germination frequency and/or vigor of conifer somatic embryos produced in vitro, the method comprising:
(a) incubating a plurality of immature conifer somatic embryos for a first incubation period in, or on, a first development media at a temperature in the range of 20° C. to 30° C.;
(b) exposing the plurality of immature conifer somatic embryos incubated in accordance with step (a) to a cold temperature in the range of 0° C. to 10° C. for a time period of at least one week; and
(c) incubating the plurality of immature conifer somatic embryos treated in accordance with step (b) for a second incubation period in, or on, a second development medium at a temperature in the range of 20° C. to 30° C.

2. The method of claim 1, wherein the cold temperature in accordance with step (b) is in the range of 1° C. to 5° C.

3. The method of claim 1, wherein the first incubation period is a time period sufficient in length for the formation of one or more cotyledonary primordia on a portion of the plurality of immature conifer somatic embryos incubated in or on the first development media.

4. The method of claim 1, wherein the first incubation period is at least six weeks.

5. The method of claim 1, wherein the first incubation period is from six to eight weeks.

6. The method of claim 1, wherein the embryos are exposed to the cold temperature for a time period of from one week to three weeks.

7. The method of claim 1, wherein the second incubation period is sufficient in length for at least a portion of the plurality of the immature conifer somatic embryos to reach anatomical maturity.

8. The method of claim 1, wherein the second incubation period is from 2 weeks to 4 weeks.

9. The method of claim 1, wherein the total incubation time for the combination of step (a), step (b) and step (c) totals a time period of at least 12 weeks.

10. The method of claim 1, further comprising the step of singulating a plurality of individual immature conifer somatic embryos prior to incubation of the embryos in accordance with step (c).

11. The method of claim 10, wherein the singulation step is carried out prior to step (b).

12. The method of claim 10, wherein the singulation step is carried out after step (b).

13. The method of claim 1, further comprising the step of culturing the embryos treated in accordance with step (c) in, or on, stratification medium at a temperature in the range of 0° C. to 10° C. for a time period of at least one week.

14. The method of claim 1, further comprising culturing the embryos treated in accordance with step (c) in, or on, a germination medium to produce germinants.

15. A method for producing mature conifer somatic embryos, comprising:
(a) culturing conifer somatic cells in, or on, an induction medium to yield embryogenic cells;
(b) culturing the embryogenic cells prepared in step (a) in, or on, a maintenance medium to multiply the embryogenic cells and form pre-cotyledonary conifer somatic embryos;
(c) culturing the pre-cotyledonary conifer somatic embryos formed in step (b) in, or on, a first development medium at a temperature in the range of 20° C. to 30° C. for a first incubation period;
(d) exposing the plurality of immature conifer somatic embryos incubated in accordance with step (c) to a cold temperature in the range of 0° C. to 10° C. for a time period of at least one week; and
(e) incubating the plurality of immature conifer somatic embryos treated in accordance with step (d) for a second incubation period in, or on, a second development medium at a temperature in the range of 20° C. to 30° C.
